(19) United States
(12) Patent Application Publication
(10) Pub. No.: US 2020/0168333 A1
(43) Pub. Date: May 28, 2020

(54) SYSTEM AND METHOD FOR CONTINUOUSLY GENERATING HEALTHCARE RECOMMENDATIONS

(71) Applicants: Kiran KALAKUNTLA, Hyderabad (IN); Srikanth SAMUDRALA, Hydearabad (IN); Amith REDDY, Hyderabad (IN)

(72) Inventors: Kiran KALAKUNTLA, Hyderabad (IN); Srikanth SAMUDRALA, Hydearabad (IN); Amith REDDY, Hyderabad (IN)

(21) Appl. No.: 16/083,901

(22) PCT Filed: Mar. 15, 2017
(86) PCT No.: PCT/IB2017/051507

§ 371 Date: Sep. 11, 2018

(30) Foreign Application Priority Data
Mar. 16, 2016 (IN) 201641009219

(51) Int. Cl. G16H 50/20; G16H 40/20; G16H 10/60; G16H 50/30

(52) U.S. Cl. CPC G16H 50/20; G16H 50/30; G16H 10/60; G16H 40/20

(57) ABSTRACT

Exemplary embodiments of the present disclosure are directed towards a system and method for continuously generating healthcare recommendations. The system comprising a health records data base unit configured to store a plurality of health records of a plurality of individuals registered with the system. A healthcare learning and recommendations engine configured to continuously learn and generate health recommendations based on the plurality of health records updated in the health records data base unit. The system further includes a feedback module configured to capture the initial set of health recommendations generated by the healthcare learning and recommendations engine.

### TECHNICAL FIELD

The present disclosure generally relates to the field of healthcare systems. More particularly, the present system and method for continuously generating healthcare recommendations.

### BACKGROUND

Presently people have more number of resources to take healthcare decisions. They have an access to multiple sources of information in order to choose the right physician for themselves and their families. Some choose a physician recommended by friends, family and other known circles. This source may not be promising when the need for a specialized medical care arises. A person needs to do exhaustive search to find out the right healthcare provider. Physician's suggestions are mostly based upon the patient's medical records. The suggestions include medical prescriptions, x-rays and other medical data depending upon one's health conditions. The suggestions can also be sourced through commercial healthcare service center. There exists a need for a provision to suggest a right healthcare provider, a right diagnostic center and a good pharmacy whose information may be accessible to the individual at any given time in a single platform.

In the light of aforementioned discussion there exists need for a system and method that would overcome or ameliorate the above mentioned disadvantages.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An objective of the present disclosure is directed towards a continuous and dynamic learning engine.

Exemplary embodiments of the present disclosure are directed towards a system and method for continuously generating healthcare recommendations, according to exemplary embodiments of the present disclosure.

According to an exemplary aspect of the present disclosure, the system includes a health records data base unit configured to store a plurality of health records of a plurality of individuals registered with the system.

According to an exemplary aspect of the present disclosure, the system includes a healthcare learning and recommendations engine configured to continuously learn and generate health recommendations based on the plurality of health records updated in the health records data base unit.

According to an exemplary aspect of the present disclosure, the system includes a feedback module configured to capture the initial set of health recommendations generated by the healthcare learning and recommendations engine.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a diagram depicting a health care recommendations environment, according to exemplary embodiments of the present disclosure.

FIG. 2A is a diagram depicting a health care recommendations system shown in FIG. 1, according to exemplary embodiments of the present disclosure.

FIG. 2B is a diagram depicting health care learning and recommendations engine, according to exemplary embodiments of the present disclosure.

FIG. 3 is a flow diagram depicting a method for continuously generating healthcare recommendations, according to exemplary embodiments of the present disclosure.

FIG. 4 is a diagrammatic representation of healthcare recommendation system of individual's home page, according to exemplary embodiments of the present disclosure.

FIG. 5A-5B is diagrammatic representation, of an overview module 418 of healthcare recommendation system, according to exemplary embodiments of the present disclosure.

FIG. 6A-6C is diagrammatic representation, of a body assessment from assessments module 420 of healthcare recommendation system, according to exemplary embodiments of the present disclosure.

FIG. 7A-7C is a diagrammatic representation, of a dental assessment from assessments module 420 of healthcare recommendation system for recommendations from experts, according to exemplary embodiments of the present disclosure.

FIG. 8 is a diagrammatic representation, of an eye assessment from assessments module 420 of healthcare recommendation system for recommendations, according to exemplary embodiments of the present disclosure.

FIG. 9 is diagrammatic representation, of a timeline module of health recommendation system, according to exemplary embodiments of the present disclosure.

FIG. 10 is a diagrammatic representation, depicting a broadcast system for healthcare recommendations, according to exemplary embodiments of the present disclosure.

FIG. 11 is a diagrammatic representation of a landing page of health recommendation system, according to exemplary embodiments of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

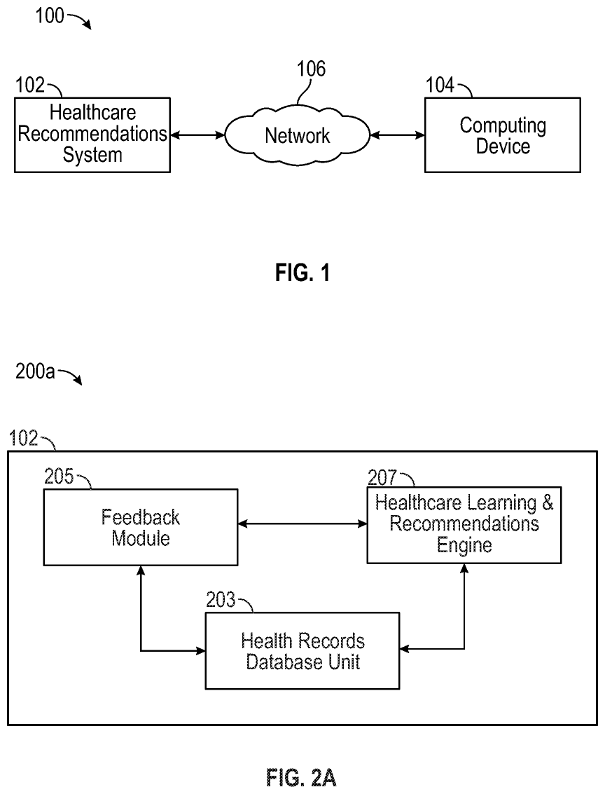

Referring to FIG. 1 is a diagram 100 depicting a health care recommendations environment, according to exemplary embodiments of the present disclosure, according to exemplary embodiments of the present disclosure. The health care recommendations environment includes a health care recommendations system 102, a computing device 104 connected over a network 106. The network 106 includes, but not limited to, an Ethernet, a local area network (LAN), or a wide area network (WAN), e.g., the internet, or a combination of networks. The computing device 104 may include a computer, a laptop, a mobile, a tablet and the like without limiting the scope of the disclosure. The health care recommendations system 102 may be configured to be a web based application and/or a mobile application using the computing device 104.

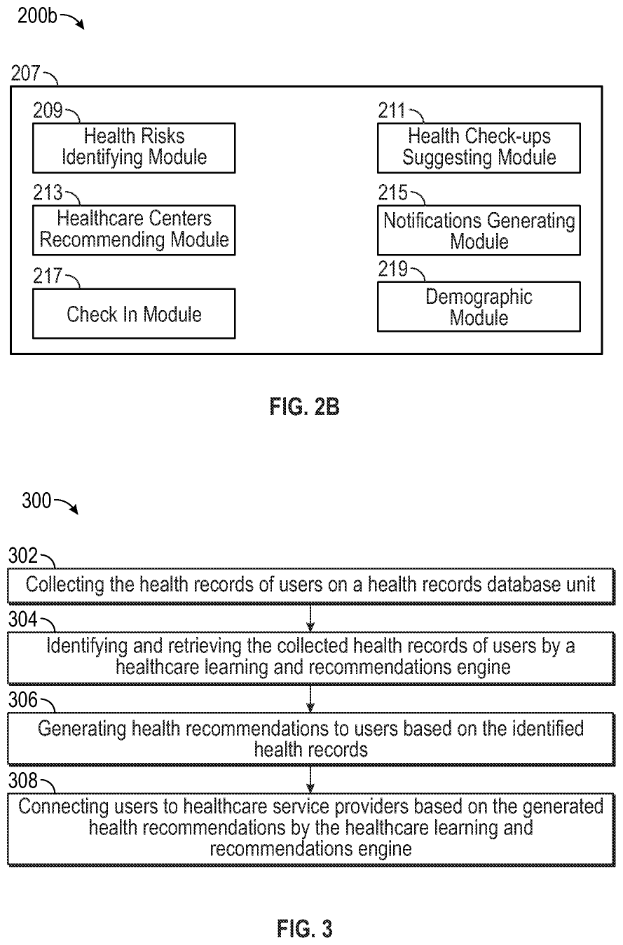

Referring to FIG. 2A is a diagram 200a depicting health care recommendations system shown in FIG. 1, according to exemplary embodiments of the present disclosure. The health care recommendations system 102 (herein after referred as system 102) may include health records data base unit 203, a feedback module 205, and a health care learning and recommendations engine 207. The system 102 may be configured to continuously learn and generate health recommendations based on the health records of updated in the health records data base unit 203. The health records data base unit 203 may be configured to store an individual individual's health records and health records of all the individuals registered with the system 102. The system 102 may be configured to individually identify and retrieve the health records of each individual for enabling the health care learning and recommendations engine 207 to continuously learn and generate health recommendations for each individual and/or in aggregation with all the registered individuals. The generated health recommendations may be customized for a individual in general, family members and combined for family members and the individual. The generated health recommendations may also be based on demographics of the individual or for a set of people with similar demographics. The demographics may include, but not limited to, age, gender, location, occupation. The health recommendations generated may include, but not limited to, medications, vaccinations, health check-ups, information related to healthcare centers, and like.

The individual's health records data base unit 203 may be populated with the individual's health records and individual's family member's health records and with the health records of all the registered individuals. The health records may include, but not limited to, medical prescriptions, diagnostic reports, and other similar medical data uploaded by the individual in online mode and/or offline mode. The health care learning and recommendations engine 207 configured to generate initial set of health care recommendations based on the health records populated in the.

The feedback module 205 configured to capture the initial set of health recommendations generated by the health care learning and recommendations engine 207 and update the individual's health records data base unit 203. The health care learning and recommendations engine 207 is configured to continuously learn the updates in the individual's health records data base unit 203 in a dynamic manner to generate further set of health recommendations. The updates to the individual's health records data base unit 203 may relate to updates in the medical records of the individual, updates in the medical records of the family members, updates of the medical records who have similar demographics as that of the individual.

Referring to FIG. 2B is a diagram depicting health care learning and recommendations engine, according to exemplary embodiments of the present disclosure. The healthcare learning and recommendations engine 207 includes a health risks identifying module 209, a health check-ups suggesting module 211, a healthcare centres recommending module 213, a notification generating module 215, a check in module 217 and a demographic module 219.

The health risks identifying module 209 may be configured to identify the health risks in the individual's health records. The health risks may include blood pressure measurement, blood sugar level determination, calculation of body mass index, measurement of triglycerides, measurement of cholesterol and like without limiting the scope of the present disclosure.

The health check-ups suggesting module 211 may be configured to suggest the health check-ups based on the health risks in the individual's health records. The health check-ups inclusive of an update from the healthcare provider on the health of the individual based on the diagnosis, vaccinations, medications, upcoming appointments and like without limiting the scope of the present disclosure. The periodic health check-ups may include dental assessment, eye assessment, body assessment, and like without limiting the scope of the present disclosure.

The healthcare service centres recommending module 213 may be configured to recommend the healthcare service centres based on the requisite health check-ups of the individual. The individual may schedule appointments directly with the healthcare service centres and get the health check-ups done through the health check-ups suggesting module 211. The service centres may include diagnostic centres, Healthcare @ home, online doctor consultations, offline doctor consultations, digital health coach, dental chains, vision chains and like without limiting the scope of the present disclosure.

The notification generating module 215 may be configured to generate notifications to individuals. The notifications may include upcoming vaccinations, medications to be taken, upcoming appointments and like without limiting the scope of the present disclosure. The notification generating module 215 may generate notifications to the individuals at a specified time.

The check in module 217 may be configured to consolidate and upload the requisite results the moment the individual checks into a healthcare centre. This enables the individual to share the diagnostic reports with the healthcare provider in a single platform.

According to non-limiting exemplary embodiments of the present disclosure the demographic module 219 may be configured to correlate the demographic profile of the population with that of the health reports. This may involve analysis of a prevalence of a certain healthcare condition based on the area, population in that area, number of males and/or females being affected by a certain health condition and the reasons thereof not limiting to the scope of the disclosure.

Referring to FIG. 3 is a flow diagram depicting a method for continuously generating healthcare recommendations, according to exemplary embodiments of the present disclosure. The method starts at step 302 by collecting the health records of individuals on a health records database unit. Identifying and retrieving the collected health records of individuals by a healthcare learning and recommendations engine, at step 304. Generating health recommendations to individuals based on the identified health records, at step 306. Connecting individuals to healthcare service providers based on the generated health recommendations by the healthcare learning and recommendations engine, at step 308.

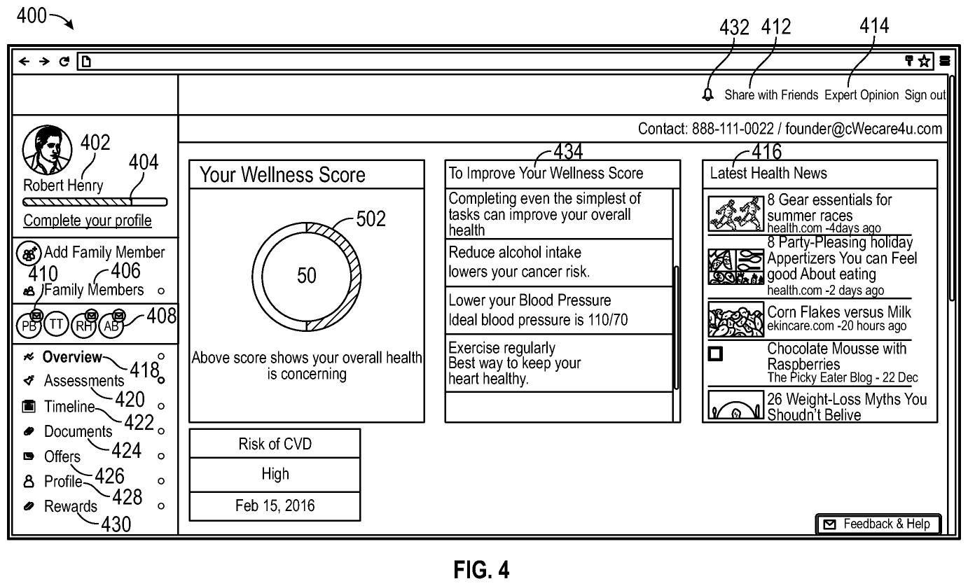

Referring to FIG. 4 is a diagrammatic representation of healthcare recommendation system of individual's home page comprising of an individual's basic details as in name of the individual 402 and completion of the profile of the percentage as a coloured bar 404. Further the individual's home page comprises of family member's details 406 inclusive of an initial alphabet of the name of the family members 408 along with a mail logo 410 which would enable the individual to know about any pending mails from the corresponding member of the family. Further the individual's home page comprises of a share with friends option 412 for sharing the opinions with friends and get goodies worth a certain amount of money and the like. The option includes allocation of a referral code and an access to a referral link for the individual. The individual's home page further comprises of an expert opinion 414 for gathering opinions from doctors based on the medical records. The individual's home page further comprises of latest health news 416 for viewing the latest health news. The individual's home page further includes; an overview module 418, an assessments module 420, a timeline module 422, documents module 424, offers module 426, a profile module 428 and rewards module 430. The assessment module 420 may include body assessment 420a, dental assessment 420b, and vision assessment 420c (not shown in FIG. 4). The individual's home page further comprises of notifications option 432 for notifying the individual. A message 434 comprising of various options to improve the wellness score is displayed. The recommendations in message 434 are displayed as per the requirement corresponding to the wellness score of the individual 502. The recommendations may include for example; “completing even the simplest of tasks can improve overall health score, reduce alcohol intake lowers your cancer risk, lower your blood pressure (ideal blood pressure is 110/70), Exercise regularly it is the best way to keep the heart healthy” and other recommendations without limiting the scope of the disclosure.

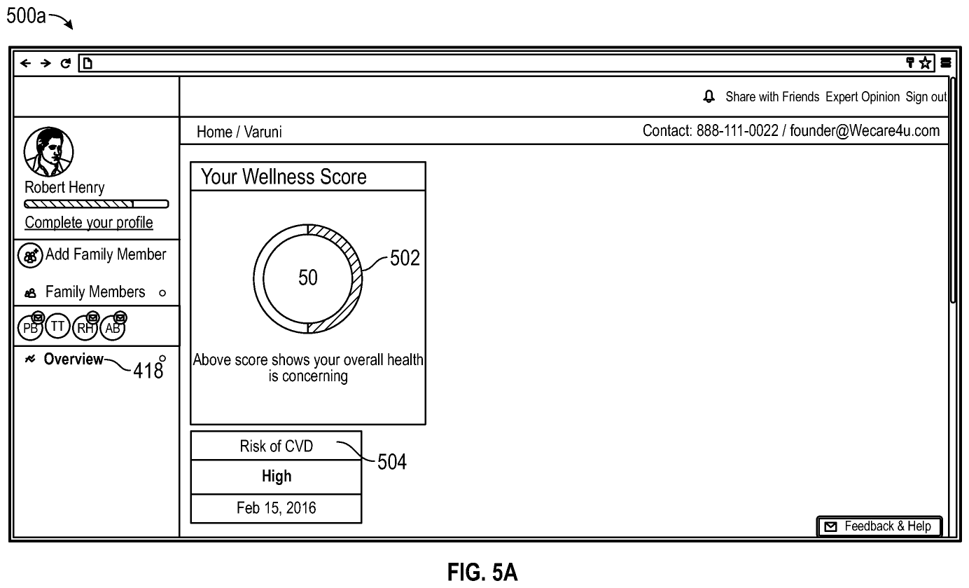

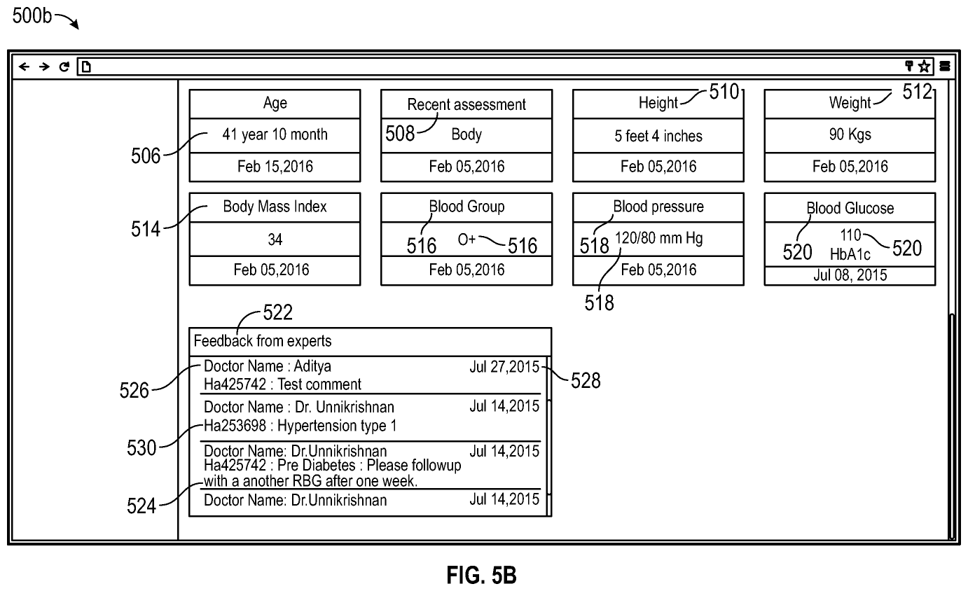

Referring to FIG. 5A is a diagrammatic representation 500a, of an overview module 418 of healthcare recommendation system, according to exemplary embodiments of the present disclosure. An overall wellness score 502 is displayed which would enable the individual to have an idea about one's general health. Further a potential risk associated with a disease 504 is displayed which would enable the individual to be alert and careful about a particular disease which the individual is at risk at a given date, according to an exemplary embodiment of the present disclosure.

Referring to FIG. 5B is a diagrammatic representation 500b of an overview module 418 of healthcare recommendation system comprising of further details of an individual's age in years and months on a given date 506, a recent assessment of one of the parameters 508, a height of the individual 510, a weight of the individual 512, a body mass index 514, a blood group 516, a blood pressure 518, a blood glucose levels 520 which may be fasting blood glucose levels, post prandial blood glucose levels, HbA1c levels and the like according to an exemplary embodiment of the present disclosure. The overview of a healthcare recommendation system further comprises of a feedback from experts 522 which may be in accordance with the results if health conditions as hypertension type 1, pre diabetes as a diagnostic recommendation and the like without limiting the scope of the present disclosure. A medication recommendation may be recommended in case of completion of diagnosis else a further diagnostic recommendation 524 for example; “please follow up with another RBG after one week” and the like may be suggested. The feedbacks from experts include other details as a name of the doctor 526, date of the test conducted 528 and an Assessment ID 530.

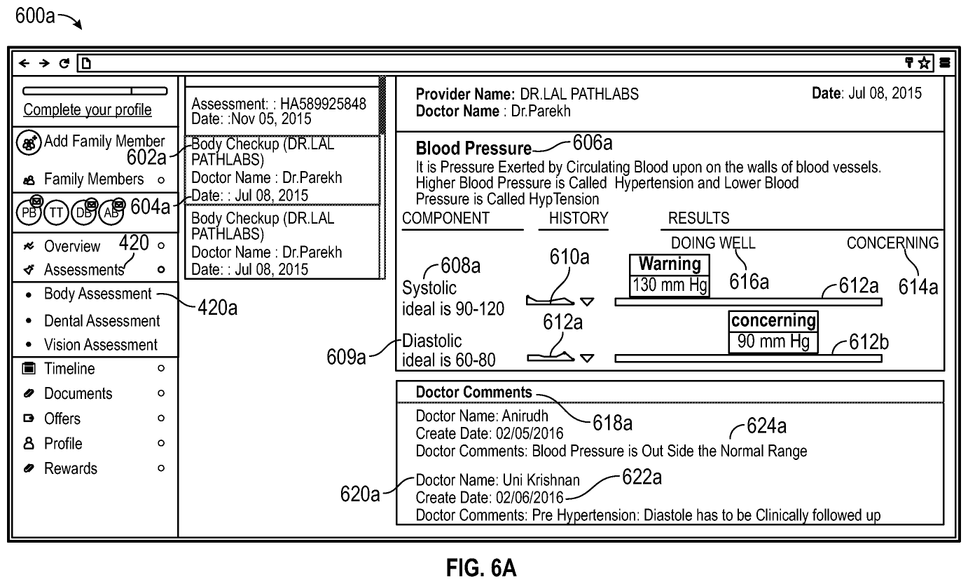

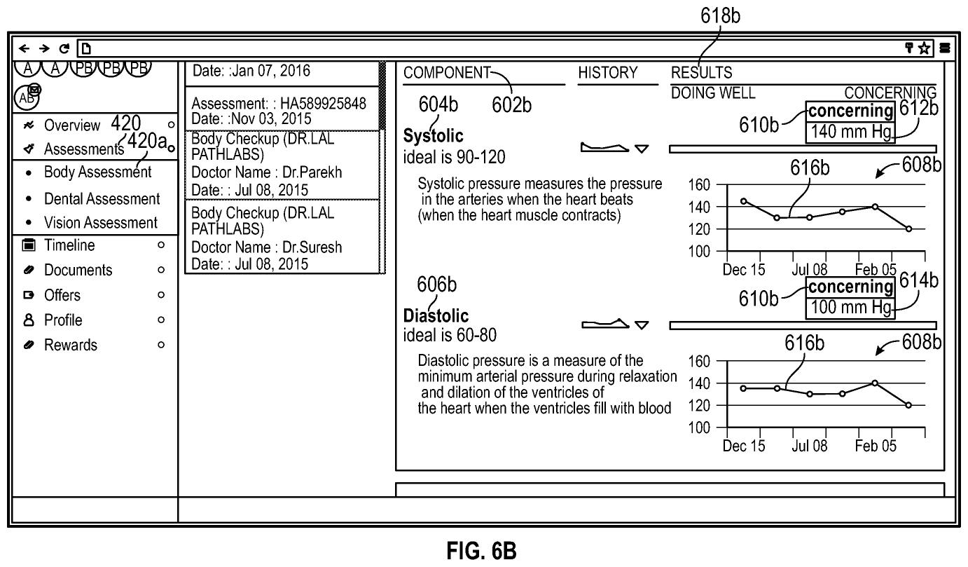

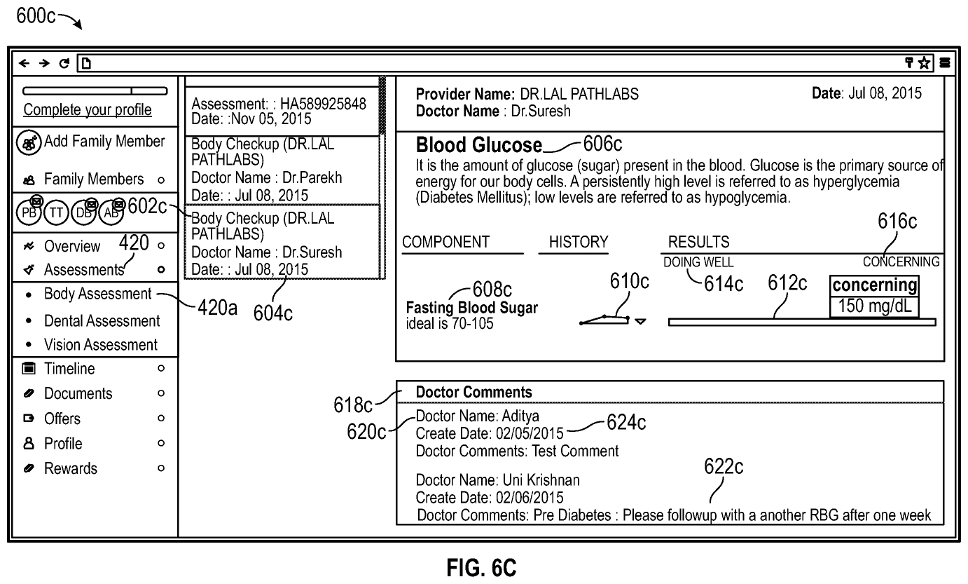

Referring to FIG. 6A is a diagrammatic representation 600a, of the body assessment 420a from assessments module 420 of healthcare recommendation system for recommendations from experts, according to exemplary embodiments of the present disclosure. The disclosure comprises of a drop down menu which includes a body checkup 602a allocated to the individual along with a date of allocation 604a. This when clicked upon further enables the display of the parameter such as blood pressure 606a and the like. Further the component of the parameter assessed may include but not limited to systolic levels 608a, and diastolic levels 609a of blood pressures which are displayed. Graphical representations 610a, 612a of a history of the levels of the components tested are displayed along with the assessments further comprising of tags as in doing well 616a and concerning 614a which is enabled to be displayed in various colour shades to enable the individual to assess based on one's parameters reading from previous tests conducted. The disclosure further comprises of doctor comments 618a involving diagnostic recommendations 624a from expert doctors based on the parameters obtained for example “blood pressure is outside the normal range, pre hypertension” and the like without limiting the scope of the disclosure. The recommendation may include other details as a name of the doctor 620a, date of the test conducted 622a.

Referring to FIG. 6B is a diagrammatic representation 600b, of the body assessment 420a from assessments module 420 (FIG. 4) of healthcare recommendation system for recommendations from experts, according to exemplary embodiments of the present disclosure. The figure includes a further representation of one of the parameter such as blood pressure 606a (FIG. 6A). The component 602b represents systolic blood pressure along with the ideal acceptable range and 604b represents diastolic blood pressure 606b along with the ideal acceptable range. A comment is displayed which enables the individual to understand a basic definition of the parameter which is being considered. For example a definition of systolic and diastolic pressure may be displayed. The history of the parameter 608b is represented graphically the representation is the month and date on which the parameter was measured versus the reading of the parameter. For example Dec 15 versus 142 mmHg for systolic blood pressure and Dec 15 versus 90 mmHg for diastolic blood pressure. The graphical involves the measurement of the parameter over a period of time involving not limiting to few readings to obtain a slope 616b to analyze the improvement or deterioration in the results of the parameter. The concerning label is represented as 610b wherein the concerning values are displayed. For example concerning levels for systolic blood pressure 140 mm Hg 612b and for diastolic blood pressure 100 mm Hg 614b. The overall results 618b as doing well, not-doing well and the like is displayed based on the overall assessment of the concerned parameter based on the graphical representation.

Referring to FIG. 6C is a diagrammatic representation 600c, of a body assessment 420a from assessments module 420 (FIG. 4) of healthcare recommendation system for recommendations from experts, according to exemplary embodiments of the present disclosure. The disclosure comprises of a drop down menu which includes a body checkup 602c allocated to the individual along with a date of allocation 604c. This when clicked upon further enables the display of a parameter not limiting to blood glucose 606c and the like. Further the component of the parameter assessed for example fasting blood sugar 608c along with the recommended ideal levels is also displayed. A graphical representation 610c of a history of the levels of the components tested 612c is displayed along with the assessments further comprising of tags as in doing well 614c and concerning 616c which is enabled to be displayed in various colour shades to enable the individual to assess based on one's parameter reading from previous tests conducted. The disclosure further includes doctor comments 618c enabling the doctor to recommend further diagnosis and/or treatment based on the obtained blood parameter for example; blood glucose. The doctor's comments may include diagnostic recommendations 622c for example: “please follow up with another RBG after one week” and the like and a medication recommendation post finalisation of the diagnosis. The doctor's comments may further include other details as a name of the doctor 620c and date of the test conducted 624c.

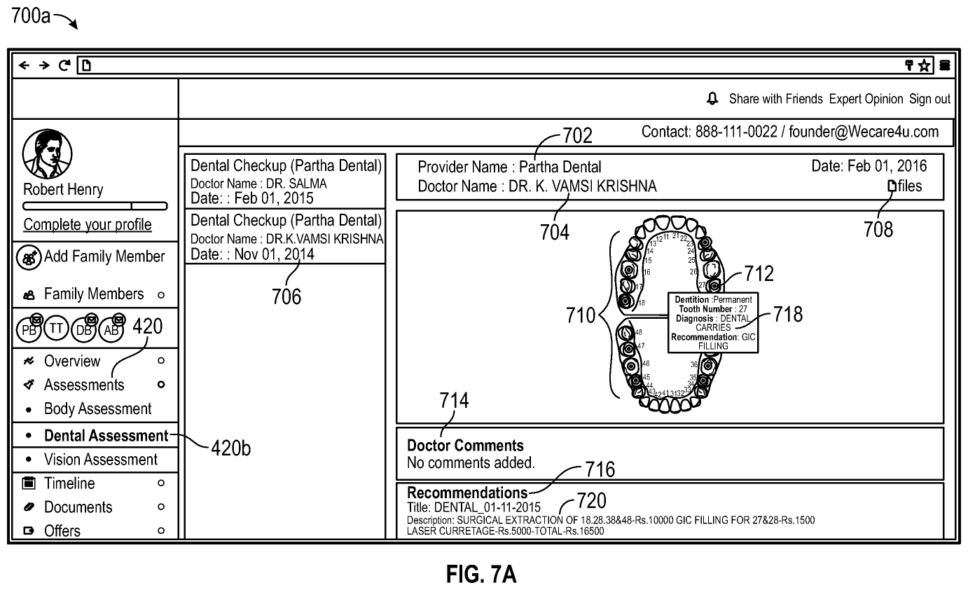

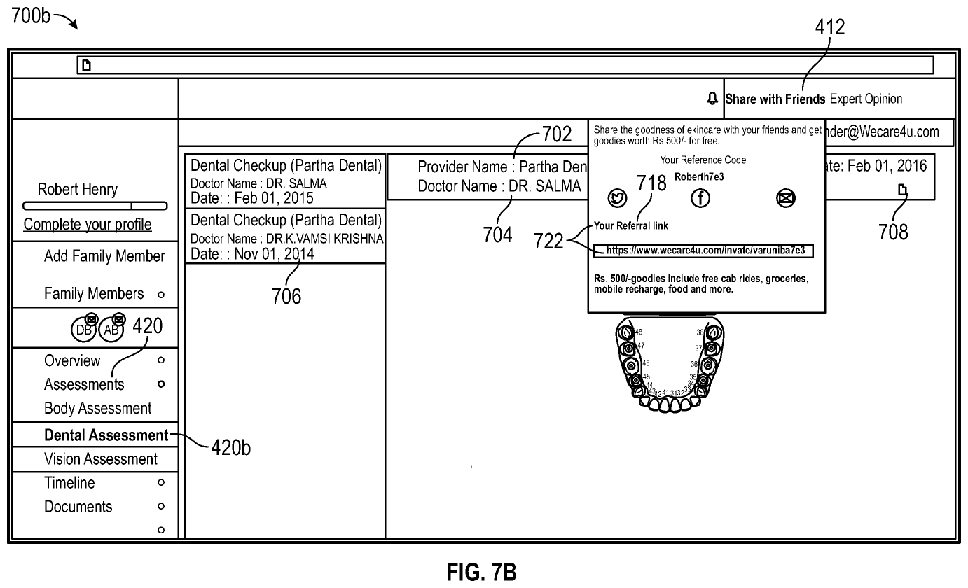

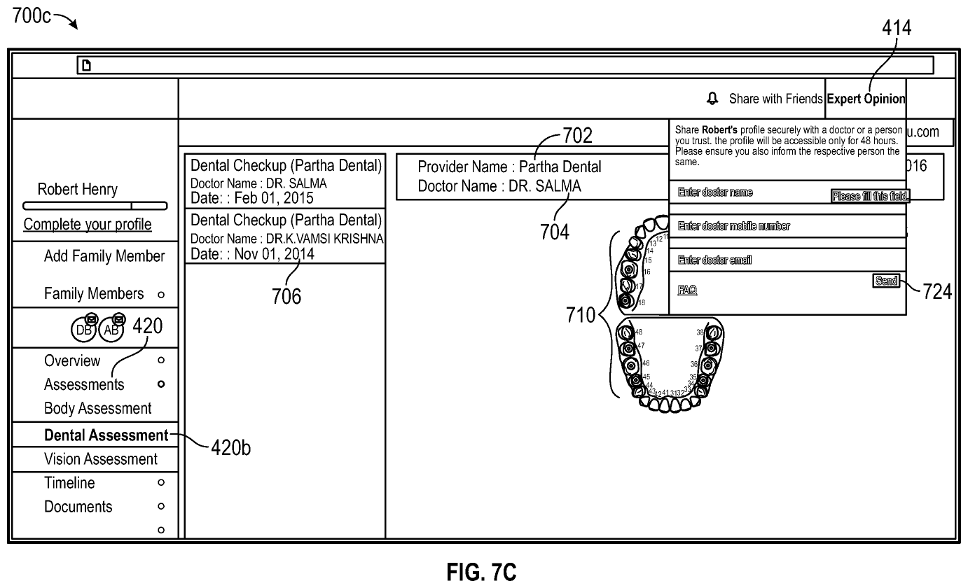

Referring to FIG. 7A-7C are diagrammatic representations 700, of a dental assessment 420b from assessments module 420 (FIG. 4) of healthcare recommendation system for recommendations from experts, according to exemplary embodiments of the present disclosure. The disclosure comprises of the name of the dent care center 702, a name of the dentist 704, a date on which a previous checkup was conducted 706 and an uploaded document of the dental report 708. Further a diagrammatic representation 710 of a dental layout is displayed. Dentation as in temporary or permanent, tooth number, diagnosis and recommendations are displayed with a dot as in 712. Further details include the doctor comments 714 and recommendations 716 are enabled by the doctors based on the dental assessment. The diagnostic recommendations 718 may include but not limited to dental carries, cavities and scaling. The recommendations may further include medical actions 720 like surgical extraction, GIC filling, titanium implants, root canal treatment and the like without limiting the scope of the disclosure. The individual may share a referral link 722 (FIG. 7B) by using the share with friend's option 412 for getting goodies worth. The individual may share his profile to experts for gathering opinions by giving the necessary credentials comprising of the doctor's name, doctor's mobile number, doctor's e mail and frequently asked questions on the expert opinion option 414. A send option 724 (FIG. 7C) may be utilized for initializing the necessary credentials on the expert opinion option 414.

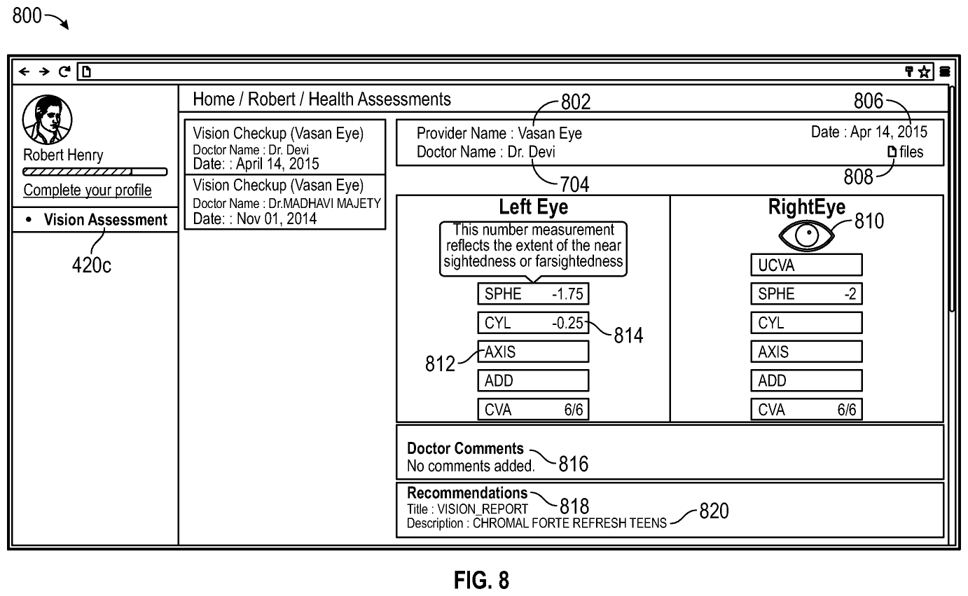

Referring to FIG. 8 is a diagrammatic representation 800, of an eye assessment 420c from assessments module 420 of healthcare recommendation system for recommendations from experts, according to exemplary embodiments of the present disclosure comprising of the details in the name of the diagnostic center 802, name of the ophthalmologist consulted 804, a date on which checkup was conducted 806 and an uploaded document of the eye report 808. Further a diagrammatic representation 810 of both left eye and the right eye is displayed. A name of each of the parameters related to an eye 812 accompanied by a small comment about the parameter and a score 814 is displayed. The disclosure further includes the doctor comments 816 and recommendations 818 which are enabled by the doctors based on the eye assessment. The diagnostic recommendations may include the description of the vision report 820. Post diagnosis a medication recommendation may be provided by the ophthalmologist for example “chromal forte refresh teens”.

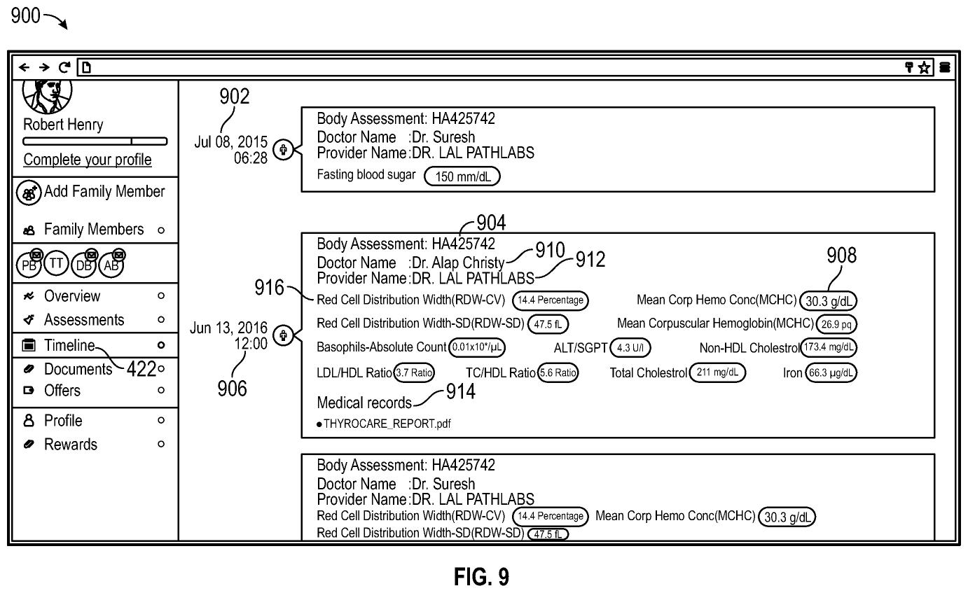

Referring to FIG. 9 is a diagrammatic representation, of a timeline module of health recommendation system, according to exemplary embodiments of the present disclosure comprising the details of series of checkups displayed along with date 902, the assessment ID 904 and a date and time during which the parameter was measured 906. A health care provider's name 910, the name of the center conducting test 912, uploaded medical records 914 are disclosed in this disclosure. Further details include but not limited to fine parameters 916 for example; Red cell distribution width, Mean Corpuscular Hemoglobin Concentration, Non-HDL cholesterol, total cholesterol, iron content and the like. The readings 908 of the fine parameters 916 are also displayed in a medically prescribed unit.

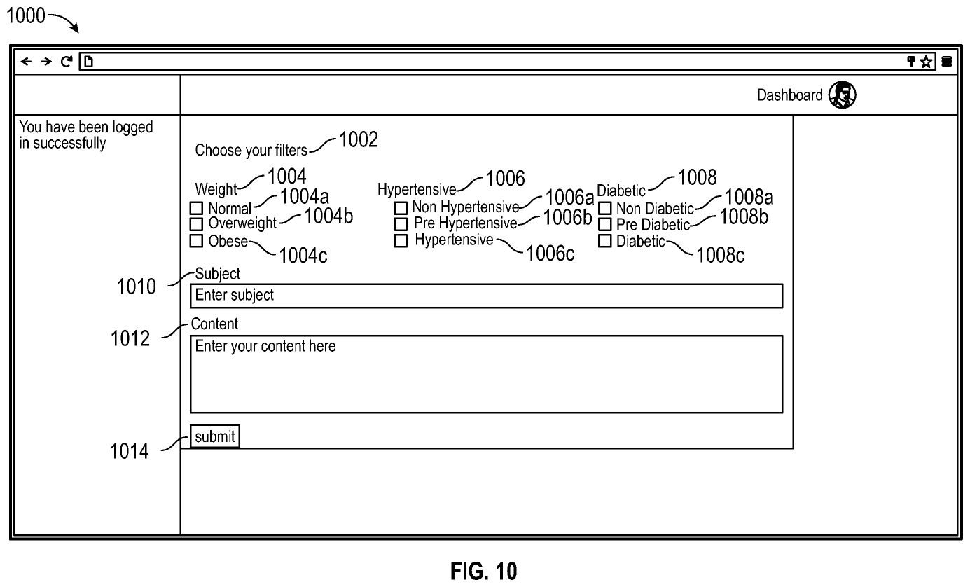

Referring to FIG. 10 is a diagrammatic representation 1000, depicting a broadcast system for healthcare recommendations, according to exemplary embodiments of the present disclosure. The broadcast system is customized messaging system wherein the individual is enabled to choose from the various filters 1002 provided. The filters may be configured to have filtering options as in weight 1004, hypertensive 1006, diabetic 1008 and the like without limiting the scope of the present disclosure. The weight which may include sub filters normal 1004a, overweight 1004b and obese 1004c. The hypertensive which may include sub filters non-hypertensive 1006a, pre hypertensive 1006b and hypertensive 1006c. The diabetic which may include sub filters non-diabetic 1008a, pre-diabetic 1008b and diabetic 1008c. Further, the individual is enabled to enter the required subject 1010 for emulation. The individual may elaborate comments in the content 1012 about the subject. A submit button 1014 enables the individual to submit the inputs.

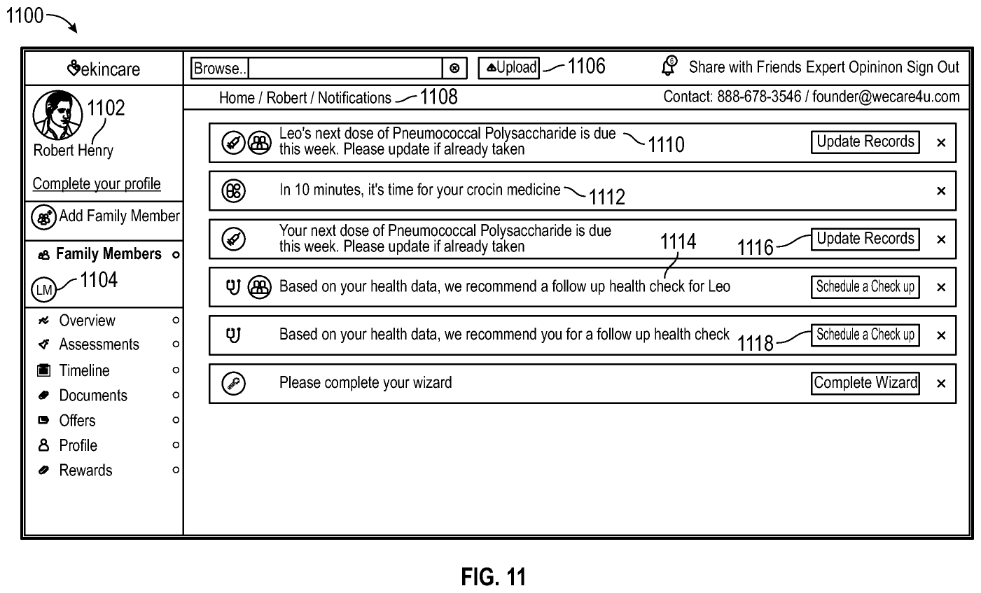

Referring to FIG. 11 is a diagrammatic representation 1100, of a landing page of health recommendation system, according to exemplary embodiments of the present disclosure. Name of the individual 1102 along with various modules 1104 is displayed. A provision to upload 1106 medical records is provided. Various recommendations for example: “Leo's nest dose of Pneumococcal Polysaccharides is due this week. Please update if already taken” 1110; similarly In 10 minutes, its ur time for your crocin medicine 1112 (indicating the time and the branded name of the medicine to be taken next); similarly based on your health data we recommend you for a follow up health check for Leo 1114 are provided along with an option to update records 1116 and schedule a checkup 1118 as per the requirement of the individual.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A system for continuously generating healthcare recommendations, comprising:
at least one health records data base unit configured to store a plurality of health records of a plurality of individuals registered with the system;
at least one healthcare learning and recommendations engine configured to continuously learn and generate health recommendations based on the plurality of health records updated in the health records data base unit; and
at least one feedback module configured to capture the initial set of health recommendations generated by the healthcare learning and recommendations engine.

2. The system of claim 1, wherein the learning and recommendations engine comprising of at least one health risks identifying module configured for identifying the health risks based on a plurality of medical histories.

3. The system of claim 1, wherein the learning and recommendations engine comprising of at least one health check-ups suggesting module configured to suggest the health check-ups based on the health risks in the plurality of health records.

4. The system of claim 1, wherein the learning and recommendations engine comprising at least one healthcare service centres recommending module configured to recommend the healthcare service centres based on the requisite health check-ups of the individual.

5. The system of claim 1, wherein the healthcare recommendation system comprising at least one notifications generating module configured to generate notifications to individuals.

6. The system of claim 1, wherein the healthcare recommendation system comprising at least one check in module configured to consolidate and upload the requisite results the moment the individual checks into a healthcare centre. This enables the individual to share the diagnostic reports with the healthcare provider in a single platform.

7. The system of claim 1, wherein the healthcare recommendation system comprising at least one demographic module configured to correlate the demographic profile of the population with that of the health reports.

8. A method for healthcare recommendations and predicting health issues, comprising:
collecting a plurality of health records of a plurality of individuals on a health records database unit;
identifying and retrieving the collected the plurality of health records of individuals by a healthcare learning and recommendations engine;
generating health recommendations to individuals based on the identified health records;
connecting individuals to a plurality of healthcare service providers based on the generated health recommendations by the healthcare learning and recommendations engine.
